(19) United States
(12) Patent Application Publication
(10) Pub. No.: US 2005/0119224 A1
(43) Pub. Date: Jun. 2, 2005

(54) Use of n-acetyl-d-glucosamine in the manufacture of pharmaceutical useful for adjuvant treatment of perianal disease

(76) Inventors: Qiwang Xu, Chongqing (CN); Junkang Liu, Chongqing (CN); Zetao Yuan, Chongqing (CN)

Correspondence Address: CHRISTIE, PARKER & HALE, LLP, PO BOX 7068, PASADENA, CA, 91109-7068, US

(21) Appl. No.: 10/469,284

(22) PCT Filed: Feb. 28, 2002
(86) PCT No.: PCT/CN02/00120

(30) Foreign Application Priority Data
Feb. 28, 2001 (CN) 01104885.9

(51) Int. Cl. A61K 31/7008

(52) U.S. Cl. 514/62

(57) ABSTRACT

The present invention has disclosed a use of N-acetyl-D-glucosamine in the manufacture of a medicine for auxiliary treatment of the peri-anal disease. Through stabilizing celluar lysosome membrance, the degree and scope of the injure extended by the release of various enzymes in the cellular lysosome are decreased; improving the healing of the injured tissues; resist the field planting of the microorganism on the traumatic surface so as to prevent the occurrence of infection. The preparation with N-acetyl-D-glucosamine as a main active component can be used in the auxiliary treatment of the peri-anal diseases, with a remarkable curative effect.

### TECHNICAL FIELD

The present invention relates to the use of N-acetyl-D-glucosane and pharmaceutical acceptable salts thereof in the manufacture of a medicament for treating the peri-anal disease.

### BACKGROUND ART

The peri-anal disease includes anal fissure, peri-anal abscess, fistula cannulas, haemorrhoids, polyp of rectum, carcinoma of the rectum and so on, the common feature of which is that there is an tissue injure in-situ, so, normally, the symptom is light, but when it acutely break out, there will be pain, red and swollen, pruritus, exudation increasing and so on, which would bring many worries and pains to the people's life and work. The treatment of these diseases needs eliminating acute oedema, lightening pain, stabilizing membrane structure, so as to prevent the inflammation to be more intensive, at the same time, it is needed to control the infection. Said disease is known as a little disease but a big problem clinically for many years. Therefore, the medicament for auxiliary treatment of peri-anal disease is needed all along in the field.

In the research of “bio-waves” theory, the present inventor has set up a bacterial wave growth model. Through researching, it is known that this wave is of its intrinsic regulation mechanism: some chemical substances are able to participate the regulation in the bio-wave process, so as to transform an abnormal periodic slow wave into a normal physiological chaotic quick wave, and this kind of substances are known as promoting wave factors. Through separating, purifying and identifying, it is determined that one of the factors is N-acetyl-D-glucosamine, the promoting wave function of which is shown in lubricating and protecting the cell. Many biochemical and physiological process of human body need the participation of the promoting wave factors, and it would lead to an abnormal state, if this kind of promoting wave factors is lacked in the living body.

N-acetyl-D-glucosamine is a chemical reagent. From the 1990's, it is continually used to treat pericementitis (WO9102530A1), microbiological infection (WO9718790A3), intestinal inflammation (WO9953929A1), cornea disease (JP10287570A2), hypertrophy of the prostate (U.S. Pat. No. 5,116,615) and so on. It is also applied in cosmetology (JP59013708A2), shampoo preparation (JP2011505A2), tissue growth regulation agent (WO/A 8 702244), and etc., but it has not been used in the manufacture of a medicament for auxiliary treatment of peri-anal disease up to now.

### CONTENTS OF THE INVENTION

The applicant of the present invention finds that N-acetyl-D-glucosamine is able to resist in situ bacterial field planting of microorganism and stabilize the membrane of cellular lysosome, so it has an auxiliary effect in the treatment of peri-anal disease.

Therefore, the present invention is related to the use of N-acetyl-D-glucosamine and pharmaceutical acceptable salt thereof in the manufacture of a medicament for auxiliary treatment of peri-anal disease.

On the other hand, the present invention is related to a method for treating peri-anal disease, including administrating to a patient who is in need thereof an effective amount of N-acetyl-D-glucosamine or pharmaceutical acceptable salts thereof.

The molecular formula of N-acetyl-D-glucosamine is C₈H₁₅NO₆, its structure is as follows: 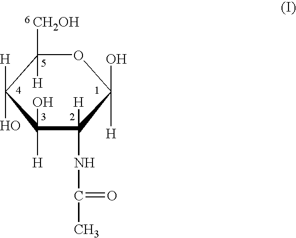
N-acetyl-D-glucosamine can be purchased in the market or prepared according to known methods. For instance, patent application WO97/31121 has disclosed a method for preparing N-acetyl-D-glucosamine from chitin by enzyme method, Japanese patent application JP63273493 has disclosed a method in which chitin is partially hydrolyzed into N-acetyl-chitose, and then it is treated with enzyme to obtain N-acetyl-D-glucosamine.

The pharmaceutical acceptable salts of N-acetyl-D-glucosamine that can be mentioned are the salts formed with pharmaceutical acceptable acids, for instance, the salts formed with inorganic acids, such as hydrochloride, hydrobromide, borate, phosphate, sulfate sulfite and hydrophosphate, and the salts formed with organic acids, such as citrate, benzoate, ascorbate, methyl sulfate, naphthalene-2-sulfonate, picrate, fumarate, maleate, malonate, oxalate, succinate, acetate, tartrate, mesylate, tosylate, isethionate, α-ketoglutarate, α-glyceryl phosphate and glucose-1-phosphate.

N-acetyl-D-glucosamine and the pharmaceutical acceptable salts thereof can be formulated in the form of liquid preparation and sprayed on a local part for auxiliary treatment of peri-anal disease, it can also be formulated into a preparation combined with other active components capable of treating peri-anal disease, so as to treat the peri-anal disease. The medicament of the present invention can be used in the sanitation of skin mucous membrane at perineum part of peri-anal for men or women, so as to relieve odor and itching, anti-inflammation, relieve pain, anti-infection, and quickly relieve and diminish the red-swollen, pruritus, pus secretion exuding condition etc. caused by anal fissure, peri-anal abscess, anal fistula, inner and outer haemorrhoids, polyp of rectum and carcinoma of the rectum. It can quickly and effectively relieve the uncomfortableness of perineum caused by seating for a long time of work.

### OPTIMAL MODE FOR CARRYING OUT THE INVENTION

The following experimental examples are used to illustrate that the compound of the present invention (the compound of formula (I)) have promoting wave function, with low toxicity, and resist in situ bacterial field planting of microorganism and stabilize the membrane of cellular lysosome.

I. Promoting Wave Test of the Compound of Formula (I)

1.1 Experimental Materials and Method:

1.1 Samples: Pure Compound of Formula (I)

1.2 Experimental Materials:

Strain: Proteus Mirabilis (which should comply with the following biological reaction characteristics: dynamics (+), urease (+), lactose (−), glucose (+), H₂S (−), phenylalanine deaminase (+).

Culture medium: modified LB culture medium (the component of the composition are: trytones of 1%, yeast extract of 0.5%, sodium chloride 1%, glucose of 0.1%, TTC of 0.002% and pH=7.2˜7.4).

1.3 Experimental Method:

The Proteus Mirabilis were inoculated at the center of LB plate, incubating at 37° C. for 9 hours, then there were concentric rings emerged, which were extended outward continually with an interval of 3 hours, and this was taken as a control; adding the compound of formula (I) with final concentration of 0.5% onto the LB plate, the Proteus Mirabilis were innoculated by the same method, cultured at 37° C., and the result showed that not only the concentric rings formed with an interval of 3 hours were emerged, comparing with the control, it can be seen that there were also many fine waves on each ring emerged.

2. Experimental Results and Evaluation:

The experiment adopts a bio-wave model which is used to research the promoting wave function of the compound of formula (I). It can be seen from the result that the compound of formula (I) was not only able to cause bacterial cell to reveal a normal bio-wave characteristic, but also cause the wave reveal finer wave mode, and these indicated that the compound of formula (I) have promoting function to bio-waves, and the promoting wave function is able to modulate the wave of the smooth muscle of intestines and the wave of the bacterial colony in intestines.

II. Toxicological Test of the Compound of Formula (I), Including:

1. acute toxicity test: including tests of administrating medicine by oral, Intravenous injection and maximum limit amount for administration;
2. Ames test;
3. micronucleus test of bone marrow cell of mouse;
4. abnormal sexual test for the sperm of mouse;
5. abnormal aberrance test for the chromosin of mouse's testis;
6. chronic lethal test;
7. subchronic toxicity (feed for 90 days) test;
8. traditional aberrance-inducing test;

The results from these tests show that, in the acute toxicity test of the compound of formula (I), the dosage more than 2 g/kg is taken, which is 300 times than the injection dosage for human being, but acute toxicosis reaction had not appeared; in the long-period toxicity test, the maximum dosage has reached up to 1 g/kg, and after the treatment and observation for four weeks, there is no toxicosis reaction yet; and in the reproduction test, the mouse was fed with routine dosages from 7 mg/kg for 3 generations, it has been proved that the compound of formula (I) has no influence on the pregnancy, birth, nurse and the growth of baby mice, so it is proved that the compound of formula (I) is a substance without toxicity.

III. Test of Resistance to Field Planting

1. Animal: Mice of Kunming Species, Provided by the 3^{rd} Military Medical University P.R. China.

2. Method:

Firstly, the animals used for experiment were infused abdominally with a suspension of Pseudomonas aeruginosa so as to make a Pseudomonas aeruginosa planting model, the mice that have Pseudomonas aeruginosa in the feces specimen cultured continuously for three days are deemed as positive. 32 mices which were positive in the experiment for making model were selected and divided into four groups, that is, three experimental groups with different dosages and one control group, eight mice in each group. The experimental groups are infused abdominally with N-acetyl-D glucosamine each day according to a schedule, while the control group is infused with saline solution. Cultivate the excrement every day, observe the saturation of Pseudomonas aeruginosa planting growth.

3. Results:

All of three dosages with high, middle and low concentrations of N-acetyl-Dglucosamine were able to effectively resist Pseudomonas aeruginosa in intestines.

IV. Test of Stabilizing Lysosome Membrane:

1. Animals: wistar rat, provided by the 3^{rd} Military Medical University P.R. China.
2. Method: setting up a rat model of endotoxin shock, at the same time, for the liver lysosome of rat, carry out a test of stabilizing membranes in vivo and in vitro. The model animals were divided into two groups, in the experimental group, N-acetyl-D-glucosamine was used to carry out the treatment, in the control group, saline solution was used to carry out the same treatment. The condition of liver lysosome mambrane was reflected by the activity of acidic phosphatase (ACP).
3. Result: the activity of free ACP in the control group and that in the experimental group are obviously different.
4. Conclusion: N-acetyl-D-glucosamine is able to effectively stabilize liver lysosome membrane of rat with endotoxin shock, and decrease the release of acidic phosphatase.
   V. Human on Trial

The experiment is based on the clinic test for the human who have suffered from peri-anal diseases, it is showed that the pharmaceutical preparations made from the compound of formula (I) are able to eliminate or relieve quickly the peri-anal uncomfortableness, pain, exudation, pruritus and the like caused by various reasons.

1-4. (canceled)

5. A method for treating perianal tissue injury, comprising:
administering to the site of the perianal tissue injury a therapeutically effective amount of a medicament that contains N-acetyl-D-glucosamine and/or a pharmaceutically acceptable salt thereof.

6. A method as recited in claim 5, wherein the medicament is in the form of a liquid.

7. A method as recited in claim 5, wherein the medicament is sprayed on the injured tissue.

8. A method as recited in claim 5, wherein the medicament is applied to a subject's perineum.

9. A method as recited in claim 5, wherein the medicament is applied to a subject's perianal mucousal membrane.
